(19) United States
(12) Patent Application Publication
(10) Pub. No.: US 2006/0217730 A1
(43) Pub. Date: Sep. 28, 2006

(54) Expandable spring loaded acetabuler reamer

(76) Inventor: Zafer Termanini, Cedar Grove, NJ (US)

Correspondence Address: Dr. Zafer Termanini, 208 Eileen Drive, Cedar Grove, NJ, 07009, US

(21) Appl. No.: 11/088,337

(22) Filed: Mar. 24, 2005

(51) Int. Cl. A61B 17/00

(52) U.S. Cl. 606/81

(57) ABSTRACT

In accordance with the present invention an improved spring-loaded expandable acetabular reamer is described, which comprises a number of convex reaming segments symmetrically located by pair around a central core of the Reamer tool. It is also an object of the present invention to provide and improved spring-loaded reaming segment, which expand faster and requires less manipulation by the operating surgeon and staff minimizing therefore the risk of infection and damage to tissue. Furthermore, introducing large size conventional acetabular reamers with rough and sharp edges through small surgical incisions will undoubtedly cause damage to the incision edge and the surrounding soft tissues, which may ultimately result in delayed wound healing. It is therefore highly desirable to provide an improved acetabular cup, which has the capacity of expanding in diameter to replace several acetabular cups thereby reducing the number of instruments used as well as shortening the time of the procedure and reducing healing time and lessen the risk of infection.

### BACKGROUND OF THE INVENTION

1. Field of Invention

The present invention relates to new and improved expandable acetabular rotary reamer and more particularly to a method and apparatus used for total hip arthroplasty.

2. Prior Art

Acetabular reamers are surgical tools, which are used during surgical procedures whereby artificial joints implants are inserted to replace articular surfaces damaged by disease. Traditionally, acetabular reamers have hemispherical surface, called reamer cup. Acetabular reamer cups are used to cut hemispherical surgical cavities in the pelvic bone. Said reamers have multiple cutting edges positioned over the spherical surface of the cup, which is attached to a tool driver. The later is driven in circular and rotation fashion by a power tool. The cutting edges would remove and grate damaged articular cartilage and continue reaming to remove a thin layer of the pelvic bone. The Reamer cup provides openings in a form of circular holes with cutting edges that are sharpened. Partly circular holes also have been used, in which the cutting edges is defined by a chord of the circle. “D” shaped holes are also known in the art and disclosed in the U.S. Pat. No. 6,730,094. Other cutting edges having different geometric shapes and configurations are also known in the art. U.S. Pat. No. 5,116,165 disclose an opening in the form of a slot. The cutting edge described in U.S. Pat. No. 5,968,049 where the cutting hole has milling teeth. More recent patents such as U.S. Pat. No. 6,428,543 describe curved outwardly cutting edges. Earlier in the art, expandable reamers as described in U.S. Pat. No. 3,702,611 utilized a reamer head having expandable three cutting blades. The expandable acetabular reaming system is described in U.S. Pat. No. 6,283,971 B1 expands using plurality of cutting blades projected from openings in the cup when an internal bladder is inflated. Other patents in the prior Art refer to Expandable head acetabular reamers such as the reamer disclosed in recent U.S. Pat. Pub. 2004/0073224, where the reaming ends may leave portions of the acetabulum unreamed. However, the use of these expandable reamers requires the operating surgeon to stop the reaming process and manually change the dial in order to obtain an accurate incremental expansion of the Reamer.

The resulting dimension and shape of the cavity cut is critical since tolerance between the cavity and the implant must be small to insure proper tight fit. This is especially critical when a “cementless” technique is used. Acetabular Reamer cups are available in multiple sizes ranging from 36 mm in diameter to about 72 mm in diameter. One surgical tray may contain in excess of 30 acetabular reamer cups. Thus, in the prior art and during a single surgical hip procedure, the surgeon may have to change a multiple cup sizes in order to obtain the appropriate size acetabular cavity.

The required exchange of multiple reamer cups has been a disadvantage of the prior art. In particular, disengaging the prior art reamer cup and exchanging it with the next size up is time-consuming and necessitates the use of a large number of reamer cup sizes during a single surgical procedure. Since time is of essence while patient is under anesthesia, it is a great advantage to reduce the operating time and the length of anesthesia. In addition, the handling of such a large number of hardware by the operating room staff may represent an increased risk for contamination and infection.

It is therefore highly desirable to provide an improved acetabular reamer cup which is capable of replacing the larger number of prior art acetabular cups which at time may exceed 30 acetabular cups. The improved acetabular expandable reamer would reduce the risk of infection and shorten the operative time since there is less hardware to handle.

### BRIEF SUMMARY OF THE INVENTION

It is an advantage of the present invention to provide a new and improved expandable acetabular reamer, which will eliminate the need during a hip surgical procedure to exchange a large number of acetabular cups in order to ream the appropriate size cavity in the acetabular bone

In the present invention, the conventional acetabular reamer cup is replaced by four convex segments that expand in a symmetric and concentric fashion around a reamer shaft with a longitudinal axis and opposite ends. Said convex segments are located at the distal end of the Reamer shaft. In the closed position, the four segment edges are in touch and form a hemispherical cup. Each of the segments is solidly attached to a guiding shaft vertically situated in the center of the segment. Said shaft glides in a tunnel located at the body of the distal part of the Reamer tool. The tunnels of two diametrically opposed cups (1 and 3) are positioned at a 45-degree angle to the longitudinal axis of the Reamer tool. In addition, these two tunnels are located in two co-adjacent planes and next to each other. When said guiding shaft is pushed outward through the tunnel, it drives the convex segment outward. When all four segments are pushed out in a concentric fashion, they will increase the diameter of the reamer cup. To allow this synchronized expansion, each convex segment is solidly attached through a hinge to the distal end of a driving lever. Said driving lever is attached proximally to a sleeve located around the central portion of the Reamer tool. The sleeve is pushed forward by a spring located within the sleeve and wrapped around the center core of the reamer tool. When said sleeve is pushed forward by the spring as described above, it will advance to the driving levers and expand the four spherical quadrant segments in a synchronized fashion similar to the opening mechanism of an umbrella. It is also the object of this invention to provide a full reaming perimeter by the cup. When expansion occurs there will be an area in the center of the cup that is not covered by the reaming segments. In order to provide hemi-circumferential reaming, there has been provision for extension tabs located at the tip of two diametrically opposed convex on segments. In the present improved invention the number of extension tabs is limited to two tabs, however in other embodiments the number of tabs can be greased by pair and may interlace to provide a full hemispherical reaming surface.

It is also an object of the invention to provide a mechanism, which allow to precisely control the synchronized expansion of the cup. Such mechanism will allow the operating surgeon, prior to the acetabular reaming process, to set a dial system that will control the maximum expansion of the convex cups.

By setting the dial to a predetermined diameter, the cups will gradually expand during the reaming process and will stop expanding when the desired diameter is reached.

In the broader aspects of the invention, there is provided a method for making a new and improved, expandable acetabular Reamer comprising multiple spherically shaped cups having a plurality of holes through the cups. Such holes have sharp edges with cutting portion that will cut and grate through the acetabular bone upon rotation of the Reamer tool by a rotary power tool.

### BRIEF DESCRIPTION OF THE DRAWINGS

The invention and the above-mentioned features will be more fully understood by reference to the following drawings.

FIG. 1 is a perspective side view of the acetabular expandable Reamer.

FIG. 2 is a perspective top view of the expandable Reamer after partial expansion.

FIG. 3 is a partially exploded fragmentary perspective side view of the improved Reamer in the closed position exposing the dial mechanism proximally and the reaming segments in closed position distally.

FIG. 4 is a top perspective view of the reaming segments in closed position.

FIG. 5 is a top perspective view of the reaming segments in the expanded position.

FIG. 6 is a cross-section view of convex reaming segment with the extension-reaming tab.

FIG. 7 is a cross-section view similar to FIG. 6 but showing spherical reaming segment without the extension reaming tab and showing the perspective driving shaft in a different location on the segment in order to allow appropriate separation.

FIG. 8 is a fragmentary top view of the distal end of the Reamer body showing the two pairs of coplanar tunnels of the improved invention as described above.

FIG. 9 is a fragmentary side view of the distal end of the Reamer body showing the two pairs of coplanar tunnels.

FIG. 10 is a cross-sectional view taken along line 50 in FIG. 3 showing the perspective component of the locking mechanism.

### DETAILED DESCRIPTION OF THE INVENTION

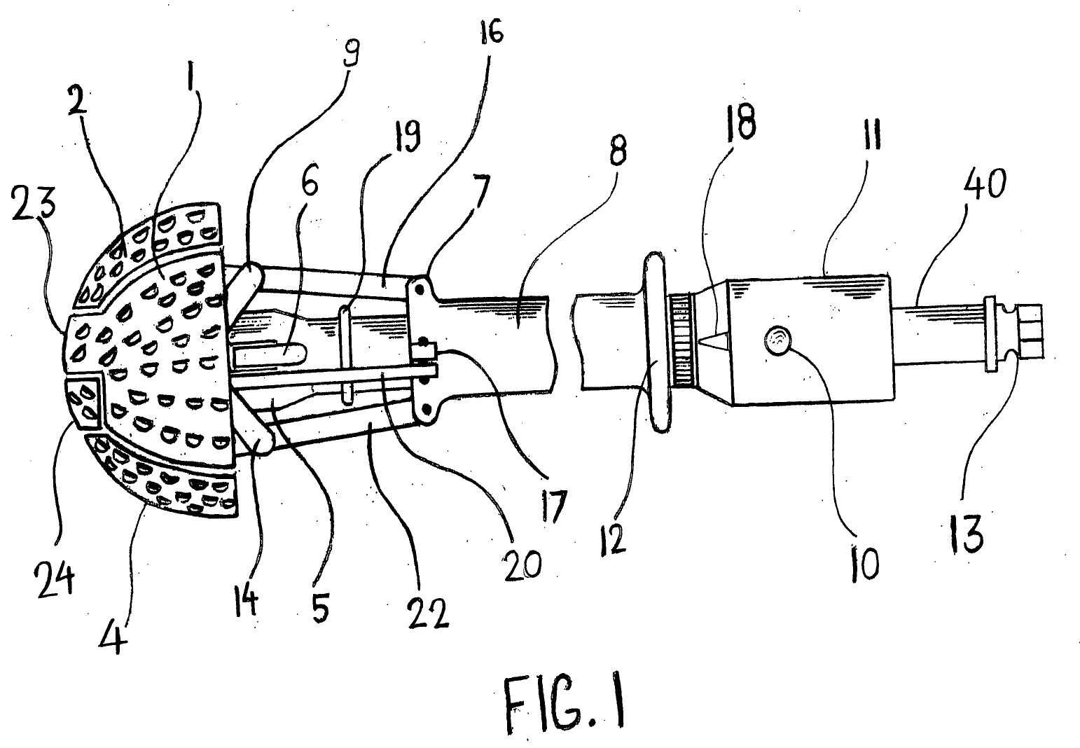

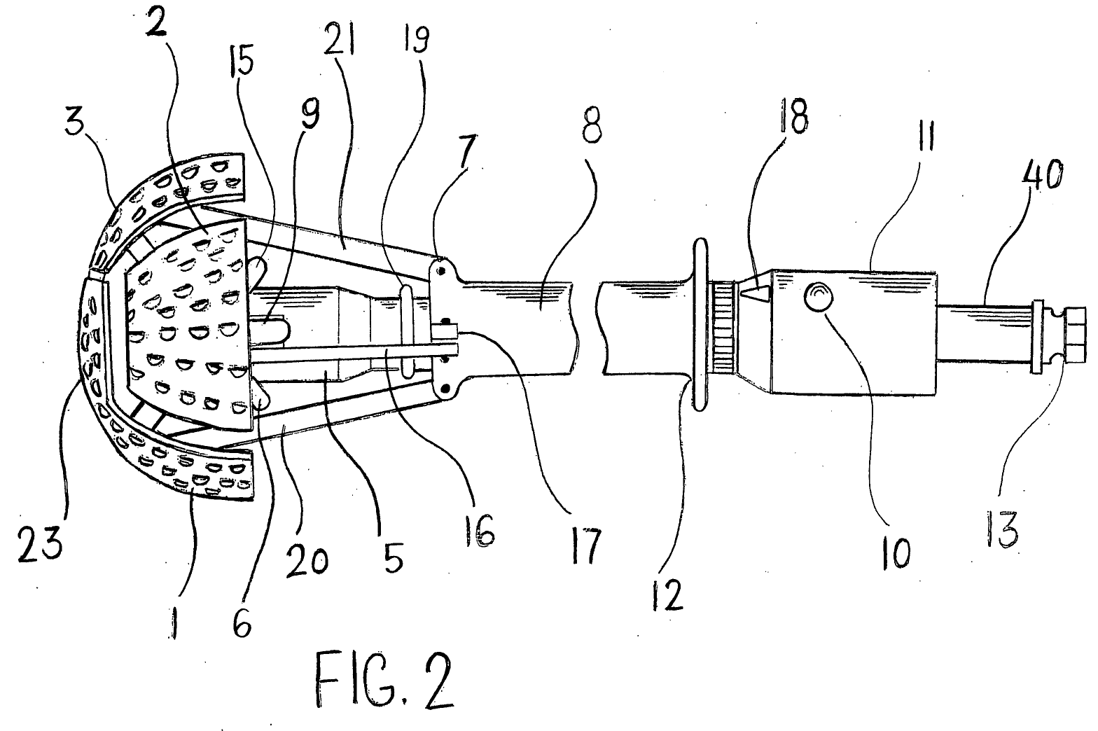

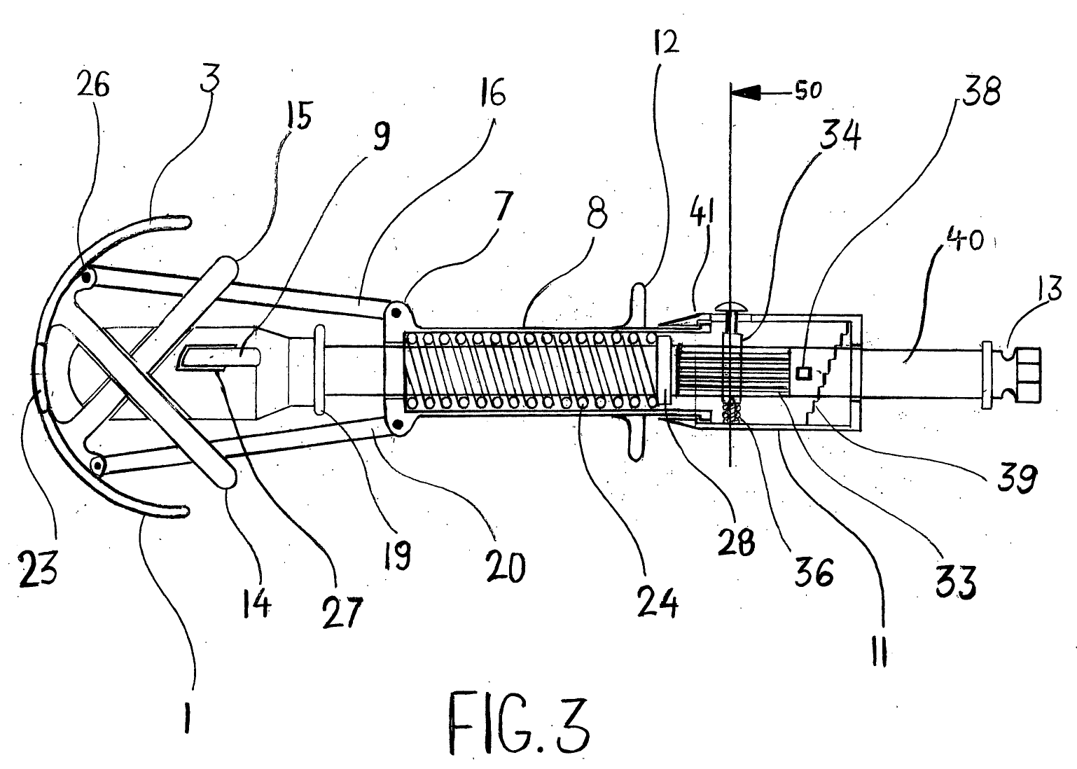

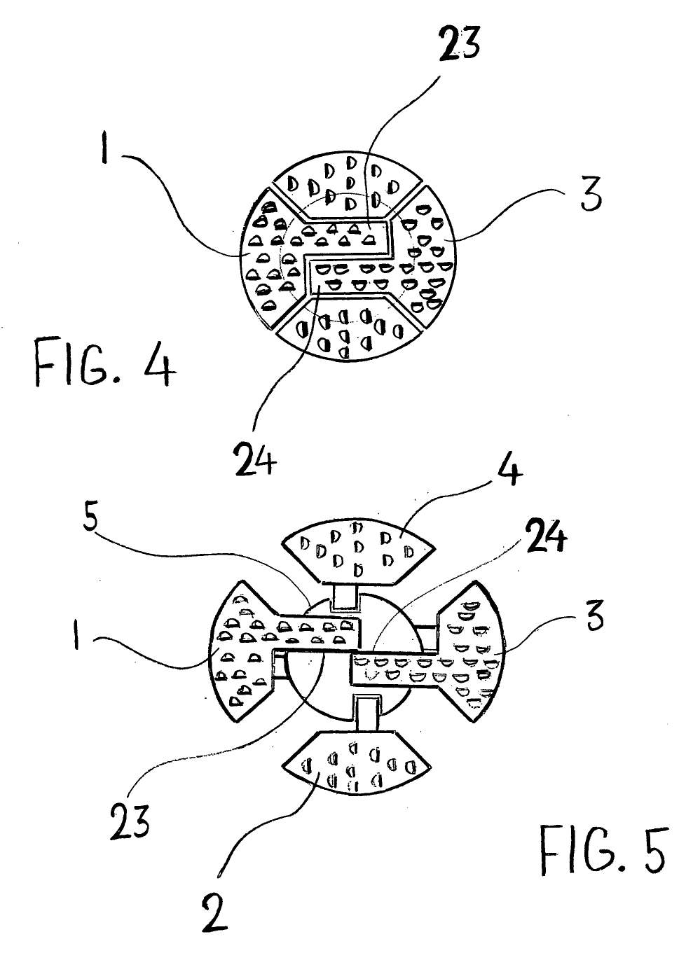

The expandable acetabular Reamer tool of this improved invention having opposed front and rear-ends 5 and 40 respectively. The front end 5 includes four arcuate and convex reaming segments as shown perspectively in FIGS. 1 and 2. The proximal end 40 is connectable to a surgical power drill known to the art by means of quick connect mechanism 13 as shown in FIG. 2 and FIG. 3.

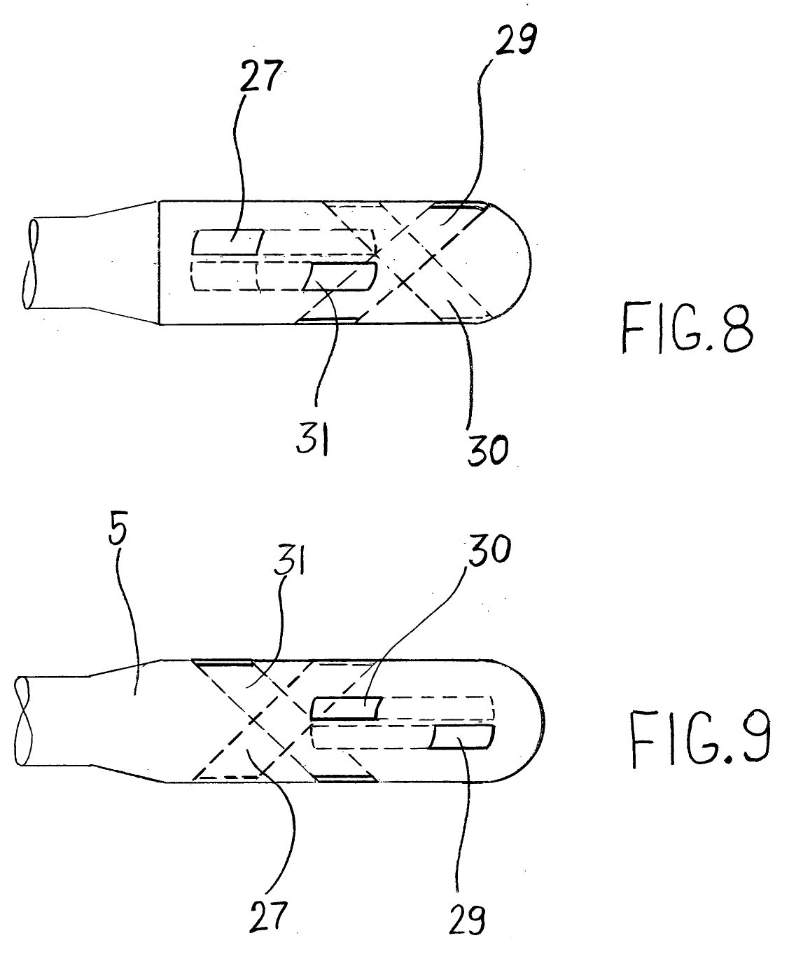

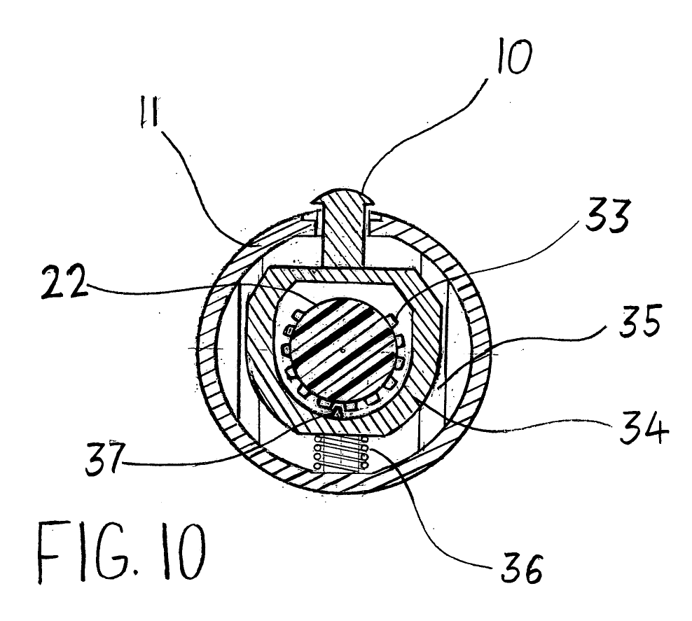

Each of the four reaming segments 1, 2, 3, and 4 is solidly attached on the concave surface to a small shaft 6, 9, 14 and 15 (guiding shaft) situated in the center of the segment. Said shafts smoothly glide into four (two pairs) of tunnels 27, 29, 30 and 31 located in the distal end 5 of the reamer tool as shown in FIG. 8 and FIG. 9. The tunnels of each pair are coplanar and have openings diametrically opposed. In addition, each pair of tunnels is situated 90° angle to each other while maintaining a 45 degree angle with the longitudinal axis of the reamer tool. None of the tunnels communicate with each other.

In order to ovoid contact between the two pairs of tunnels the proximal and distal pairs are placed at two different levels along the central axis of the core of the distal end 5 of the Reamer tool. In this fashion there is appropriate separation of the two pairs of tunnel and would avoid impingement.

Based on the above, it is clear that in order to compensate for the separation of the tunnels, the guiding and driving shafts of the small reaming segments are attached in an eccentric fashion and closer to the edge of the small segment, as shown in FIG. 7. Referring back to FIG. 3 it becomes evident that the distal tunnels are used for the large reaming segments (having extension tabs) and the proximal tunnels are used for the guiding shafts of the small reaming segment. Furthermore, each of the four segmental cups is attached on the convex surface to a driver lever 16, 20, 21, and 22 through a hinge 26. Said driver lever is proximally connected to the gliding sleeve 8 through hinge 7 and 17 as shown in FIG. 3. The gliding sleeve 8 provide a collet 12 which may be grabbed by the operating surgeon to help pull the gliding sleeve rearward. By doing so, the Reamer segments will reapproximate together into the closed position and form a hemispherical cup of reduced diameter which will facilitate ingress and egress of the reaming tool from small surgical incisions.

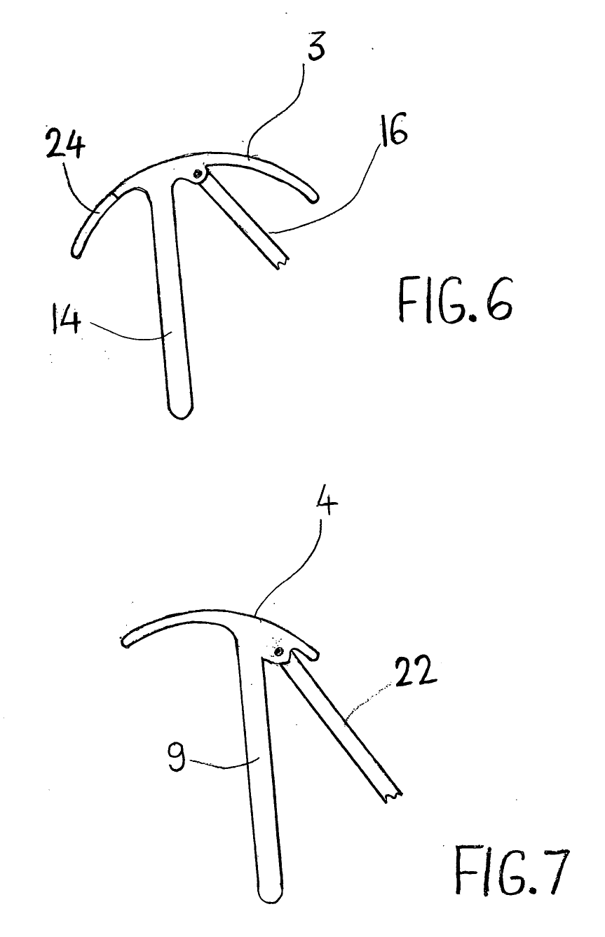

The gliding sleeve 8 is driven forward by spring 24 located within the gliding sleeve. Said spring 24 is wrapped around the central shaft of the Reamer tool and resting proximally against stop 28. When allowed, the gliding sleeve will continue to move forward until it rests against stop 19. In doing so, said sleeve will push driving shafts 16, 20, 21 and 22 forward. It is to be noted as shown in FIG. 6 and FIG. 7 that there are two small segments 2, 4 and two large segments 1, 3. Only the large segments have extension tabs 23 and 24.

The purpose of these extension tabs is to provide reaming in the center of the reamer as expansion progress. Without the tabs, there will be a non-desirable central portion or a core of bone that is left unreamed, resulting into a non-congruent spherical surface. This will hinder the placement of the acetabular prosthesis into the reamed cavity.

The forward movement of the driving shafts collectively is thereby transmitted to the convex reaming segments, which will be advanced outwards and expend in a concentric and symmetric fashion, as shown in FIG. 2 and FIG. 5. The outward movement and expansion of the reaming cups is constrained and guided by the geometric position of the guiding shafts 6, 9, 14, and 15, gliding smoothly in tunnels 15, 27, 35, and 36 located in the distal portion of the reamer tool as shown in FIG. 8 and FIG. 9. The expansion of the reaming cups is actuated by the forward movement of the sleeve 8 which will continue to glide at forward until it is limited by stop 19.

It is further the object of this invention to provide a mechanism by which the surgeon is able to control the expansion of the reaming cups to a predetermined size. Referring now to FIG. 3, which depicts the mechanism controlling the expansion, located in the proximal end of the driver tool. Said mechanism comprises a cylindrical housing 11 which contains a circular and progressive step stop 39 fixed to the inner surface of said cylindrical housing 11. Said housing 11 can be the freely rotated around the shaft of the Reamer tool 40. It is however attached to the gliding sleeve 8 through a shoulder collar 41 located at its distal portion. The gliding sleeve and the cylindrical housing will travel as one unit when pulled rearward by the operating surgeon. By pulling rearward the gliding sleeve 8, the operating surgeon would then be able to rotate the housing unit 11 and set dial 18 to a predetermined and desirable diameter of the reaming cups. When the gliding sleeve 8 is allowed to travel forward, it will travel along with housing 11 until it stopped by pin 38 which will then rest against the chosen step of the circular Progressive stop 39.

It is further the object of this invention to provide a locking mechanism, which will allow the longitudinal travel of housing 11 and prevent the accidental or inadvertent rotation of the housing 11. Said locking mechanism comprises a locking ring 34 traveling into vertical slots 35 located within the housing 11.

The locking ring 34 can be depressed by button 10 against a spring 36 located within housing 11 and situated diametrically opposed to button 10. Furthermore, the lock 34 contains a locking knob 37 which will sit into longitudinal grooves 33 milled around the surface of the Reamer tool shaft 40. Said longitudinal grooves run parallel to the longitudinal axis of the reamer tool.

When locking knob 37 is engaged in a grooves it will only allow housing 11 to travel along the direction and path of that particular groove thereby eliminating any accidental turning of housing 11 during the reaming operation. Each of the longitudinal grooves correspond to a specific position of the indicator 18 on the indicia which represent a specific but the everyday assess an RBI minute cup size expansion.

In a different embodiment of this improved acetabular Reamer. The number of the reaming segments may vary from two large semi circumferential cups to four or more segments. Subsequently, adjustment of the number of driver and guiding shafts should be made. In other embodiment of this invention, the stopping pin 38 with the located on the inner surface of housing 11 and the step ratchet 39 would be placed centrally around the central shaft 40 of the reaming tool.

Having described preferred embodiments of the invention with reference to the accompanying drawings, it is to be understood that the invention is not limited to those precise embodiments and various changes and modifications could be effected by one skilled in the art without departing from the spirit or scope of the present invention, as defined in the appended claims.

I claim:

1. A rotary acetabular bone Reamer tool for use in hip surgery comprising:
a Shaft having a longitudinal axis and opposite ends;
a proximal end engageable and disengageable from a rotable driving power tool such as surgical power driver;
a distal end comprising a plurality of reaming convex segments having plurality of holes with cutting edges to grate the acetabular cavity in the pelvic bone.

2. The bone Reamer tool of claim 1 wherein the reaming process is accomplished by means of four reaming convex segments.

3. The bone Reamer tool of claim 2 wherein the reaming convex segments are each attached to a guiding shaft and a driving shaft.

4. The bone Reamer tool of claim 3 wherein the four reaming segments having two small segments and two large segments, where said small and said large segments are diametrically opposed to form a complete hemispherical cup.

5. The bone Reamer tool of claim 4 wherein the two large reaming segments have each an extension tab with convex surface having plurality of holes with cutting edges to grate the acetabular cavity in the pelvic bone.

6. The extension tabs of claim 5 are located side-by-side in the closed position and slide apart but remain adjacent to each other when the four reaming segments are in the expanded position.

7. The Reamer tool of claim 6 wherein the four guiding shafts glide smoothly into two pairs of tunnels located in a coplanar fashion at the distal end of said the Reamer tool.

8. The Reamer tool of claim 7 wherein the two pairs of tunnels are coplanar and situated at 90 degree to each other and 45° to the longitudinal axis of said Reamer tool where the two pairs of tunnels are located distally and the other two pair are located proximally.

9. The two pairs of tunnels of claim 8 are independent of each other and do not communicate.

10. The driver shafts of claim 3 are attached with hinges to the concave surface of the reaming segments distally and to the gliding sleeve proximally.

11. The driver levers and the guiding shafts of claim 3 wherein the location of the driver and guiding shaft of the two small reaming segments are eccentrically located and closer to the free edge of that segment in order to allow appropriate spacing of the guiding shafts and their respective tunnels.

12. The Reamer tool of claim 4 wherein the gliding sleeve is slidably located around the central core of the driver tool where said gliding sleeve is driven forward by a spring wrapped around the central core of the driver tool but within the gliding sleeve.

13. The gliding sleeve of claim 4 is attached at its distal portion to four driver levers whereby the gliding sleeve when moved up forward will push said driving levers forward.

14. Said driver levers of claim 6 when pushed forward will drive the four convex reaming segments outward in a concentric and symmetric fashion similar to the opening of an umbrella.

15. Reamer tool of claim 1 comprises an adjustable locking mechanism located at the proximal end of said reamer tool, said locking mechanism is situated within the locking handle and will allow the operating surgeon to control the amount of expansion of the four convex reaming segments.

16. The locking mechanism of claim 15 comprising multiple longitudinal grooves milled on the surface of the center core of the Reamer tool where each groove corresponds to a size on the expansion dial located on the outside of the locking mechanism.

17. The locking mechanism of claim 15 comprises a circular locking ring actuated by a spring located at its lower portion and controlled by a button located at the top which is protruding at the surface of the locking handle, when said button is depressed by the operating surgeon, will displace the locking ring downward and disengage the locking pin from the longitudinal grooves thereby allowing the handle to rotate coaxially around the central core of the reaming tool further allowing the operating surgeon to move the pointer to the chosen number on the indicia.

18. The locking mechanism of claim 17 where said locking button controls the locking pin, engageable in a chosen longitudinal groove thereby fixing the handle and the locking mechanism at that specific position.

19. The locking mechanism of claim 15 comprises a circular and progressive stop situated over the inner surface of the locking handle.

20. The locking mechanism of claim 15 comprises a stopping pin located on the body of the central core of the reaming tool where said stopping pin will come to rest against a circular and progressive stop, thereby preventing the locking handle as well as the gliding sleeve to the slide forward and expand the reaming segment.
